(19) United States
(12) Patent Application Publication
(10) Pub. No.: US 2022/0143075 A1
(43) Pub. Date: May 12, 2022

(54) OPHTHALMIC PHARMACEUTICAL COMPOSITIONS AND METHODS FOR TREATING OCULAR SURFACE DISEASE

(71) Applicant: SURFACE OPHTHALMICS, INC., Pleasanton, CA (US)

(72) Inventors: Dennis Elias Saadeh, Nashville, TN (US); Kamran Hosseini, Pleasanton, CA (US)

(21) Appl. No.: 17/582,627

(22) Filed: Jan. 24, 2022

Related U.S. Application Data
(63) Continuation-in-part of application No. 16/650,071, filed on Mar. 24, 2020.
(60) Provisional application No. 62/562,809, filed on Sep. 25, 2017.

(51) Int. Cl. A61K 31/737; A61K 31/573; A61K 31/343; A61K 9/00; A61K 31/721; A61K 9/08; A61K 47/10; A61K 47/18; A61K 47/02

(52) U.S. Cl. CPC A61K 31/737; A61K 31/573; A61K 31/343; A61K 9/0048; A61K 47/02; A61K 9/08; A61K 47/10; A61K 47/183; A61K 31/721

(57) ABSTRACT

Pharmaceutical compositions including betamethasone sodium phosphate and mycophenolic acid and their use in the treatment of ocular surface disease.

### CROSS REFERENCE TO RELATED APPLICATION(S)

This application is a continuation in part of U.S. Ser. No. 16/650,071, which is a US national phase under 35 U.S.C. § 371 of international patent application no. PCT/US2018/052185, filed Sep. 21, 2018, which claims the benefit of priority under 35 U.S.C. § 119(e) of U.S. Ser. No. 62/562,809, filed Sep. 25, 2017, the entire content of each of which is incorporated herein by reference.

### FIELD OF THE INVENTION

The present invention relates generally to the field of ophthalmology and more specifically to compositions and methods for treating, mitigating, and/or preventing ocular surface disease, such as dry eye disease in mammals.

### BACKGROUND

Ocular surface disease, such as dry eye disease, is an ophthalmic condition that manifests itself in symptoms of discomfort and visual disturbance as a result of decreased tear production and is characterized by a dysfunction of one or more components of the tear film, the latter being stable in the absence of this disease. Tear deficiency may be caused by poor production of tears as a result of age, hormonal changes, various autoimmune diseases, and other factors, and may also be a side effect of certain medications, such as beta-blockers, antidepressants, antihistamines, etc. Ocular surface disease can also occur following ocular surgery. A normal stable condition of the tear film resulting in normal tear secretion is important for the lubrication and maintenance of the refractive surface of the eye.

Ocular surface disease may afflict an individual and vision may be substantially impaired with varying degrees of severity, ranging from burning sensation, a feeling of dryness and persistent irritation up to substantial impairment of vision in more severe cases. Therefore, a variety of approaches have been developed for treatment and therapy of such diseases. Typically, the majority of patients with an ocular surface disease are prescribed or recommended artificial tears. Other methods and devices that are also often recommended include scrubs, drops, inserts, plugs or lid compresses. These products typically include immunologic agents, autologous compounded serum, mucin producing agents and/or lubricants. While some such remedies do exist, and may provide some relief in some cases, in many other instances they are insufficient or too expensive. Accordingly, it is desirable to have better alternative compositions.

### BRIEF SUMMARY OF THE INVENTION

The present disclosure addresses the above-described deficiencies in the treatment of ocular surface disease and provides related benefits. In particular, pharmaceutical compositions suitable for treatment, prevention, and/or alleviation of ocular surface disease that can achieve positive patient outcomes while being free of drawbacks and deficiencies of existing formulations are described, as well as methods of fabricating and administering the same.

According to one embodiment of the invention, a pharmaceutical composition is provided for topical administration to an eye, the composition including betamethasone sodium phosphate at a concentration of 0.01% w/w to 0.05% w/w; mycophenolic acid at a concentration of 0.05% w/w to 0.50% w/w; and a pharmaceutically acceptable carrier for topical administration to the eye.

In some embodiments, the composition consists essentially of the betamethasone sodium phosphate at a concentration of 0.01% w/w to 0.05% w/w; mycophenolic acid at a concentration of 0.05% w/w to 0.50% w/w; and the pharmaceutically acceptable carrier, which preferably includes added lubrication for the eye. In related embodiments the pharmaceutical composition can also include a glycosaminoglycan, such as chondroitin sulfate and/or a deturgescent agent, such as dextran. Preferably chondroitin sulfate is provided at a concentration of 0.1% to 5.0% w/w and dextran is provided at a concentration of 0.1% to 5.0% w/w.

According to other embodiments of the invention, methods are provided for using the above-mentioned compositions for treating, preventing, and/or alleviating various forms of an ocular surface disease such as keratoconjunctivitis sicca, episodic dry eye disease, chronic dry eye disease, recalcitrant dry eye disease, age-related dry eye, neurotrophic ocular surface disease, and blepharitis. In such embodiments, a therapeutically effective amount of the pharmaceutical composition is administered topically to the subject's eye that is suffering or at risk of suffering the ocular surface disease. In some embodiments, the ocular disease is caused or worsened by ocular surgery at least temporarily and thus, the composition may be provided as a post-surgical ocular treatment.

In other embodiments, methods for treating, preventing, and/or alleviating various forms of an ocular surface disease are provided, which include topically administering to an eye of a subject suffering from the ocular surface disease, a therapeutically effective amount of a pharmaceutical composition comprising betamethasone sodium phosphate at a concentration of 0.01% w/w to 0.05% w/w. In some embodiments, the ocular disease is caused or worsened by ocular surgery at least temporarily and thus, the composition may be provided as a post-surgical ocular treatment. In some embodiments, the composition consists essentially of the betamethasone sodium phosphate at a concentration of 0.01% w/w to 0.05% w/w; and the pharmaceutically acceptable carrier provides added lubrication for the eye. However, in other embodiments the pharmaceutical composition can include a glycosaminoglycan, such as chondroitin sulfate and/or a deturgescent agent, such as dextran. Preferably chondroitin sulfate is provided at a concentration of 0.1% to 5.0% w/w and dextran is provided at a concentration of 0.1% to 5.0% w/w.

In other embodiments, methods for treating, preventing, and/or alleviating various forms of an ocular surface disease are provided, which include topically administering to an eye of a subject suffering from the ocular surface disease, a therapeutically effective amount of a pharmaceutical composition comprising mycophenolic acid at a concentration of 0.05% w/w to 0.50% w/w. In some embodiments, the ocular disease is caused or worsened by ocular surgery at least temporarily and thus, the composition may be provided as a post-surgical ocular treatment. In some embodiments, the composition consists essentially of the mycophenolic acid at a concentration of 0.05% w/w to 0.50% w/w; and the pharmaceutically acceptable carrier provides added lubrication for the eye. However, in other embodiments the pharmaceutical composition can include a glycosaminoglycan, such as chondroitin sulfate and/or a deturgescent agent, such as dextran. Preferably chondroitin sulfate is provided at a concentration of 0.1% to 5.0% w/w and dextran is provided at a concentration of 0.1% to 5.0% w/w

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees, depending on the context. Whenever it appears herein, a numerical range such as “1 to 20” refers to each integer in the given range; i.e., meaning only 1, only 2, only 3, etc., up to and including only 20.

The term “salt” refers to an ionic compound which is a product of the neutralization reaction of an acid and a base.

The terms “solvate” and “hydrate” are used herein to indicate that a compound or a substance is physically or chemically associated with a solvent for “solvates” such as water (for “hydrates”).

The term “mycophenolic acid” or “MPA” refers to the compound having the IUPAC name 6-(4-hydroxy-6-methoxy-7-methyl-3-oxo-1H-2-benzofuran-5-yl)-4-methylhex-4-enoic acid and the following chemical structure:

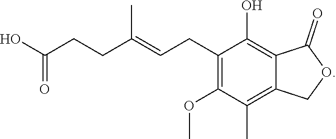 [SMILES: COc1c(C)c2c(c(O)c1C/C=C(\C)CCC(=O)O)C(=O)OC2]

The term “cyclosporine” refers to the compound having the IUPAC name (3 S,6S,9S,12R,15S,18S,21S,24S,30S,33 S)-30-ethyl-33-[(E,1R,2R)-1-hydroxy-2-methylhex-4-enyl]-1,4,7,10,12,15,19,25,28-nonamethyl-6,9,18,24-tetrakis(2-methylpropyl)-3,21-di(propan-2-yl)-1,4,7,10,13,16,19,22,25,28,31-undecazacyclotritriacontane-2,5,8,11,14,17,20,23,26,29,32-undecone and the following chemical structure:

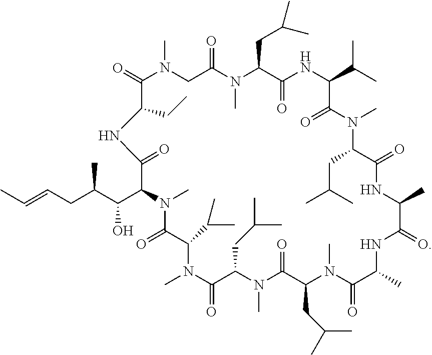 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

The term “corticosteroid” refers to any steroid hormone, both produced synthetically and obtained from the adrenal cortex of vertebrates (inclusive of both glucocorticoids and mineralocorticoids) and belonging to a sub-genus of steroids that are derivatives of corticosterone, the latter having the chemical structure:

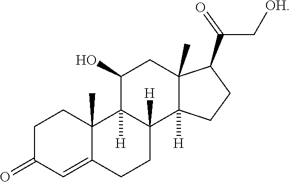

The term “tacrolimus,” also known as fujimycin or FK506, refers to an compound having the IUPAC name (−)-(3S,4R,5S,8R,9E,12S,14S,15R,16S,18R,26aS)-8-allyl-5,6,8,11,12,13,14,15,16,17,18,19,24,25,26,26a-hexadecahydro-5,19-dihydroxy-3-{(E)-2-[(1R,3R,4R)-4-hydroxy-3-methylcyclohexyl]-1-methylvinyl}-14,16-dimethoxy-4, 10,12,18-tetramethyl-15,19-epoxy-3H-pyrido[2,1-c][1,4]oxaazacyclotricosane-1,7,20,21(4H,23H)-tetrone, and the following chemical structure:

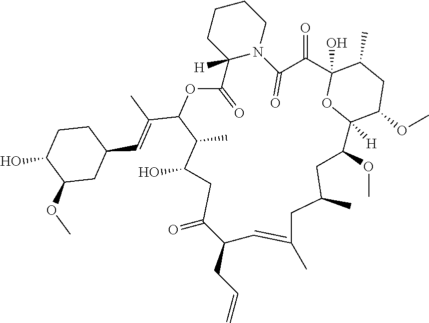 [SMILES: C=CC[C@@H]1/C=C(\C)C[C@H](C)C[C@H](OC)[C@H]2O[C@@](O)(C(=O)C(=O)N3CCCC[C@H]3C(=O)OC(/C(C)=C/[C@@H]3CC[C@@H](O)[C@H](OC)C3)[C@H](C)[C@@H](O)CC1=O)[C@H](C)C[C@@H]2OC]

The term “albumin” refers to any not glycosylated proteins found in blood plasma.

The term “plasma” refers to blood plasma, i.e., a liquid that comprises extracellular matrix of blood cells.

The term “platelet-rich plasma” refers to a concentrate derived from blood, from which red blood cells have been removed.

The term “serum” refers to a protein-rich liquid obtained in the process of coagulation of blood, i.e., plasma from which clotting proteins have been removed.

The term “glycosaminoglycan” refers to any unbranched polysaccharide comprising a repeating disaccharide unit.

The term “deturgescent agent” refers to a compound that is capable of maintaining the stroma of the cornea of the eye in a state of relative dehydration to an extent necessary to ensure the transparency of the cornea.

The term “ocular surface disease” (including “dry eye”, “dry eye syndrome” and “dry eye disease”) is defined as one or several conditions associated with, or caused by, either decreased or insufficient tear production or increased or excessive tear film evaporation, or both, and characterized by redness, itching, and burning of the eye. An ocular surface disease is further defined as being inclusive of keratoconjunctivitis sicca, episodic dry eye disease, chronic dry eye disease, recalcitrant dry eye disease, age-related dry eye, neurotrophic ocular surface disease, and blepharitis. Ocular surface diseases such as at least some of those listed above also commonly occur following ocular surgery procedures.

The term “pharmaceutical composition” is defined as a chemical or a biological compound or substance, or a mixture or combination of two or more such compounds or substances, intended for use in the medical diagnosis, cure, treatment, or prevention of disease or pathology.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable,” when used in the context of a carrier, is defined as a carrier, whether diluent or excipient, that is compatible with the other ingredients of the formulation and not deleterious to the recipient thereof.

The terms “administration of a composition” or “administering a composition” are defined to include an act of providing a compound or pharmaceutical composition of the invention to the subject in need of treatment.

### B. Embodiments of the Invention

The present application discloses pharmaceutical compositions useful for treating, preventing, and/or alleviating an ocular surface disease. In various embodiments, the compositions include 0.01% w/w to 0.05% w/w betamethasone sodium phosphate and 0.05% to 0.50% w/w mycophenolic acid, and may include compounds such as a glycosaminoglycan (e.g. chondroitin sulfate), and/or a deturgescent agent (e.g. dextran). The compositions further include a carrier such as de-ionized water and/or balanced salt solution. Principal components of the composition include the following.

Betamethasone, also referred to herein as betamethasone sodium phosphate, is in a class of medications called corticosteroids. Eye drops and eye ointments containing betamethasone are offered under the brand names BETRICIN, CELUDEX, EYEBET, and METHASOL and include betamethasone at 0.1% or higher. Betamethasone-based eye drops are known to treat short term inflammatory eye conditions, such as to relieve inflammation, redness and irritation caused by hay fever and allergic rhinitis. Common side effects of betamethasone-based eye drops are irritation, burning, stinging, itching, and blurred or clouded vision. Within the present invention, it has been surprisingly found that betamethasone can be used at a significantly lower concentration, in particular from 0.01% to 0.05% w/w when used in the treatment of dry eye disease, such as after undergoing ocular surgery. This lower concentration significantly reduces the side effects of steroids commonly found at conventional dosages.

Mycophenolic acid is an immunosuppressant, and thus lowers the activity of the immune system. In particular, it prevents the proliferation of T-cells, lymphocytes and the formation of antibodies from B-cells. It is most commonly known for its use in organ transplantation, such as after kidney, heart and liver transplantation. It is also used to treat autoimmune conditions such as Crohn's disease and lupus. Surprisingly, it has been found that mycophenolic acid, together with the substantially decreased dosage of betamethasone sodium phosphate, provides an effective treatment for ocular surface disease, such as dry eye disease, when applied topically to the eye of subjects. In particular, it was surprisingly found that when combining betamethasone sodium phosphate at a concentration from 0.01.% to 0.05% w/w and mycophenolic acid at a concentration from 0.05% to 0.50% w/w in a pharmaceutical composition formulated as eye drops for topical use on the eye, an effective treatment for ocular surface disease, such as dry eye disease was achieved. This has been further identified as particularly useful as a treatment after undergoing ocular surgery. This combination has been found to eliminate in some subjects or at least significantly reduce unwanted treatment effects found with higher concentrations of compounds like betamethasone sodium phosphate.

The pharmaceutical compositions disclosed herein are formulated for use as eye drops for topical administration to the eye and thus include a pharmaceutically acceptable carrier that is acceptable for administration to the eye. While the pharmaceutically acceptable carrier can include simple saline solutions, preferably the carrier includes a lubricating agent. Nonlimiting examples of suitable lubricating agents include glycerol, glycerin, or glycerine. Alternatively, if desired, another compound may be used as a lubricating agent in addition to, or instead of, glycerol, glycerin, or glycerine, if desired. Non-limiting examples of acceptable lubricating agent(s) that may be so used include any of: polyvinyl pyrrolidone, sorbitol, polyethylene glycol, hydroxypropylmethyl cellulose, carboxy propylmethyl cellulose, and polyvinyl acetate. As generally guidance, lubricants can be provided at 0.1% w/w to 5.0% w/w, such as between 1.0% w/w and 4.0% w/w, for example 1.0% w/w. In preferred embodiments, the lubricating agent is 0.1% glycerol.

In some embodiments the pharmaceutical composition consists essentially of betamethasone sodium phosphate, mycophenolic acid and the carrier, preferably with lubricating agent; however, in other embodiments, the pharmaceutical compositions can also include at least one glycosaminoglycan. It can be theorized, without firm commitment to any particular or specific mechanism, that glycosaminoglycans may be useful in protecting endothelial and epithelial cells which are subject to exposure to trauma, and/or to promote the growth of such cells. Non-limiting examples of glycosaminoglycan(s) that may be used include: chondroitin, chondroitin sulfate, dermatan sulfate, dermatin sulfate, heparin sulfate, heparan sulfate, keratin sulfate, keratan sulfate, or hyaluronic acid. The preferred glycosaminoglycan is chondroitin sulfate.

The total contents of the glycosaminoglycan(s) in the composition expressed as the mass concentration may be from 0.1% w/w to 5.0% w/w, such as from 0.2% w/w to 4.0% w/w, for example 0.25% w/w.

The pharmaceutical compositions with or without the glycosaminoglycan can also include at least one deturgescent agent. The preferred deturgescent agent is dextran. However, non-limiting examples of other acceptable deturgescent agent(s) that may be used in addition to, or instead of, dextran include any of: dextran sulfate, sodium chloride (NaCl), potassium chloride (KCl), dextrose, and sucrose. While such deturgescent agents are typically used to provide dehydration for stroma of the cornea of the eye, as defined above, unexpectedly, deturgescent agents used in the compositions disclosed herein are also beneficial for improving outcomes in the process of treatment of various surface ocular diseases such as dry eye disease.

The total contents of the deturgescent agent(s) in the composition expressed as the mass concentration may be from 0.1% w/w to 5.0% w/w, such as from 0.2% w/w to 4.0% w/w, for example 0.25% w/w.

In some embodiments, the composition may also include one or more antioxidants selected from the group consisting of ascorbic acid derivatives such as ascorbic acid, erythorbic acid, and sodium ascorbate; Thiol derivatives such as thioglycerol, cysteine, acetylcysteine, cystine, dithioerythreitol, dithiothreitol, glutathione; Tocopherols; butylated hydroxvanisol (BHA); butylated hydroxytoluene (BHT); sulfurous acid salts such as sodium sulfate, sodium bisulfite, acetone sodium bisulfite, sodium metabisulfite, sodium sulfite, sodium formaldehyde sulfoxylate, and sodium thiosulfate; and nordihydroguaiaretic acid.

In related embodiments of the present invention, pharmaceutical compositions having fewer than the above-recited components are provided for treating, preventing, and/or alleviating an ocular surface disease. In various embodiments, the compositions may comprises or consist essentially of betamethasone sodium phosphate at 0.01% to 0.05% w/w and a pharmaceutically acceptable carrier that is suited for topical administration to the eye. In various embodiments, the composition may include one or more of: mycophenolic acid, tacrolimus, cyclosporine, albumin, plasma, platelet-rich plasma, serum, and pharmaceutically acceptable salts, hydrates, solvates, esters thereof or derivatives or analogs thereof.

In those embodiments where the compositions comprise, consist of, or consist essentially of betamethasone sodium phosphate with or without mycophenolic acid or a pharmaceutically acceptable salt or derivative thereof, the formulation can be present in a solution either as a part of a polycarbophil-based formulation or as a part of a non-polycarbophil-based formulation. In various embodiments, the total content of the mycophenolic acid in the composition expressed as the mass concentration may be from 0.05% w/w to 0.5% w/w, such as from 0.1% w/w to 0.5% w/w, for example, 0.1%, 0.2%, 0.3%. 0.4% or 0.5% w/w, while the total content of betamethasone sodium phosphate in the composition expressed as the mass concentration may be from 0.01% w/w to 0.05% w/w, such as 0.01%, 0.02%, 0.03%, 0.04% and 0.05% w/w.

In various embodiments, the compositions may comprise or consist essentially of mycophenolic acid at 0.05% to 0.50% w/w and a pharmaceutically acceptable carrier that is suited for topical administration to the eye.

In some embodiments, any of the above compositions may also include one or more antioxidants selected from the group consisting of ascorbic acid derivatives such as ascorbic acid, erythorbic acid, and sodium ascorbate; Thiol derivatives such as thioglycerol, cysteine, acetylcysteine, cystine, dithioerythreitol, dithiothreitol, glutathione; Tocopherols; butylated hydroxyanisol (BHA); butylated hydroxytoluene (BHT); sulfurous acid salts such as sodium sulfate, sodium bisulfite, acetone sodium bisulfite, sodium metabisulfite, sodium sulfite, sodium formaldehyde sulfoxylate, and sodium thiosulfate; and nordihydroguaiaretic acid.

As mentioned above, in addition to any of the above-described components, the compositions also include a carrier. In some embodiments, the carrier comprises pure de-ionized water. In other embodiments, the carrier includes a balanced salt solution known to those having ordinary skill in the art. In yet other embodiments, the carrier may, in addition to water and/or a balanced salt solution, further optionally contain some other products, such as one or several pharmaceutically acceptable excipient(s). In some embodiments, if an excipient is used, it can be a non-ionic polyoxyethlene-polyoxypropylene block copolymer having the following general structure:

HO—(CH₂—CH₂—O)ₓ—(C₃H₆—O_{y}—(CH₂—CH₂—O)ₓ—H,

wherein x is an integer having the value of at least 8 and y is an integer having the value of at least 38.

If a non-ionic polyoxyethlene-polyoxypropylene block copolymer is used as an excipient, its contents in the overall composition may be from 0.01 mass % and 20.0 mass %, such as from 0.2 mass % to 15 mass %, for example, 0.2 mass %.

One non-limiting example of a specific non-ionic polyoxyethlene-polyoxypropylene block copolymer that can be used as a solubilizing and stabilizing agent in the pharmaceutical compositions of the instant invention is the product known under the trade name POLOXAMER 407 (poly(ethylene glycol)-block-poly(propylene glycol)-block-poly(ethylene glycol)), with the molecular weight of the polyoxypropylene portion of about 4,000 Daltons, about a 70% polyoxyethylene content, the overall molecular weight of between about 9,840 Daltons and about 14,600 Daltons.

Another type of product that can be used in the excipient portion of the pharmaceutical formulation may be water-soluble methylcellulose and hydroxypropyl methylcellulose polymers, such as METHOCEL family of products, for example, a hydroxypropyl methylcellulose product METHOCEL E4M. The compositions may also contain a quantity of preservative(s) such as benzalkonium chloride, if desired.

Yet another type of product that can be used in the excipient portion of the pharmaceutical formulation may be a polycarbophil polymer product (i.e., a polymeric product based on polyacrylic acid cross-linked with divinyl glycol) which is available under a variety of trade names such as FIBERCON, EQUALACTIN, KONSYL FIVER, etc. If a polycarbophil product is used it may also be present as a part of mycophenolic acid solution, as mentioned above.

Finally, the ophthalmic compositions will typically have an osmolarity from 100 to 500 milliosmoles per liter (mOsm/L), such as from 150 mOsm/L to 450 mOsm/L, for example, from 200 mOsm/L to 400 mOsm/L. A tonicity modulating agent, such as sodium chloride, may also be used in the compositions.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Alternatively, a two- or multiple-batch method(s) may be used if desired, where each component of the pharmaceutical formulation can be combined in separate container followed by combining the contents of each container. The resulting product may then be transferred into single dose vials, capped, sealed, autoclaved and shaken until cool. Finally, a complete sterility and endotoxin analysis may be performed on the product according to commonly used methods known to those having ordinary skill in the art.

Pharmaceutical compositions prepared as described above can be used for treating, preventing, and/or alleviating an ocular surface disease, i.e., including, without limitation, keratoconjunctivitis sicca, episodic dry eye disease, chronic dry eye disease, recalcitrant dry eye disease, age-related dry eye, neurotrophic ocular surface disease, and blepharitis. The pharmaceutical compositions are also particularly useful for administration after ocular surgery, when such conditions are likely to occur. The compositions bring about a significant relief to the sufferers of such diseases. Among other benefits, the uncomfortable “stinging” or “burning” feeling in the eye that is routinely experienced in formulations including steroids is eliminated or at least significantly decreased after the composition has been administered; which in view of at least the conventional use of steroid formulations is a highly unexpected effect.

To this end, a method of treating dry eye disease is provided, which includes: administering to a subject suffering from dry eye disease, a therapeutically effective amount of a pharmaceutical composition including or consisting essentially of betamethasone sodium phosphate at a concentration of 0.01% w/w to 0.05% w/w; mycophenolic acid at a concentration of 0.05% w/w to 0.50% w/w; and a pharmaceutically acceptable carrier for topical administration to the eye. In some embodiments, the pharmaceutical composition consists essentially of the betamethasone sodium phosphate, the mycophenolic acid and the pharmaceutically acceptable carrier, preferably including a lubricant, for topical administration to the eye. In other embodiments, the pharmaceutical composition includes one or more of a glycosaminoglycan (e.g. chondroitin sulfate from 0.1% to 5.0% w/w); a deturgescent agent (e.g. dextran from 0.1% to 5.0% w/w); or other compound substantially as described above or below.

In furtherance of the above, the following pharmaceutical compositions were tested against 0.05% cyclosporine ophthalmic emulsion (RESTASIS) and 5% lifitegrast ophthalmic solution (XIIDRA®) as a treatment for dry eye disease: a composition containing 0.01% w/w betamethasone sodium phosphate and 0.3% w/w mycophenolic acid, compositions containing either 0.1% or 0.3% w/w mycophenolic acid, and 0.01% w/w betamethasone sodium phosphate. Subjects were dosed twice daily (BID) for 84 days (12 weeks).

Still further a related method treating dry eye disease is provided, which includes: administering to a subject suffering from dry eye disease, a therapeutically effective amount of a pharmaceutical composition including or consisting essentially of betamethasone sodium phosphate at a concentration of 0.01% w/w to 0.05% w/w; and a pharmaceutically acceptable carrier for topical administration to the eye. In some embodiments, the pharmaceutical composition consists essentially of the betamethasone sodium phosphate and the pharmaceutically acceptable carrier, preferably including a lubricant, for topical administration to the eye. In other embodiments, the pharmaceutical composition includes one or more of a glycosaminoglycan (e.g. chondroitin sulfate from 0.1% to 5.0% w/w); a deturgescent agent (e.g. dextran from 0.1% to 5.0% w/w); or other compound substantially as described above or below. The pharmaceutical may further include mycophenolic acid.

Still further a related method treating dry eye disease is provided, which includes: administering to a subject suffering from dry eye disease, a therapeutically effective amount of a pharmaceutical composition including or consisting essentially of mycophenolic acid at a concentration of 0.05% w/w to 0.50% w/w; and a pharmaceutically acceptable carrier for topical administration to the eye. In some embodiments, the pharmaceutical composition consists essentially of the mycophenolic acid and the pharmaceutically acceptable carrier, preferably including a lubricant, for topical administration to the eye. In other embodiments, the pharmaceutical composition includes one or more of a glycosaminoglycan (e.g. chondroitin sulfate from 0.1% to 5.0% w/w); a deturgescent agent (e.g. dextran from 0.1% to 5.0% w/w); or other compound substantially as described above or below.

In another related embodiment, a post-surgical treatment method is provided, which includes topically administering to an eye of a subject that has undergone ocular surgery, a therapeutically effective amount of a pharmaceutical composition, which includes betamethasone sodium phosphate at a concentration from 0.01% to 0.05% w/w; mycophenolic acid at a concentration from 0.05% w/w to 0.50% w/w; and a pharmaceutically acceptable carrier for topical administration to the eye. In some embodiments, the pharmaceutical composition consists essentially of the betamethasone sodium phosphate, myophenolic acid, and a pharmaceutically acceptable carrier for topical administration to the eye. In other embodiments, the pharmaceutical composition includes one or more of a glycosaminoglycan (e.g. chondroitin sulfate from 0.1% to 5.0% w/w); a deturgescent agent (e.g. dextran from 0.1% to 5.0% w/w); or other compound substantially as described above or below.

In still another related embodiment, a post-surgical treatment method is provided, which includes topically administering to an eye of a subject that has undergone ocular surgery, a therapeutically effective amount of a pharmaceutical composition, which includes betamethasone sodium phosphate at a concentration from 0.01% to 0.05% w/w and a pharmaceutically acceptable carrier for topical administration to the eye. In some embodiments, the pharmaceutical composition consists essentially of the betamethasone sodium phosphate, and the pharmaceutically acceptable carrier, preferably including a lubricant, for topical administration to the eye. In other embodiments, the pharmaceutical composition includes one or more of a glycosaminoglycan (e.g. chondroitin sulfate from 0.1% to 5.0% w/w); a deturgescent agent (e.g. dextran from 0.1% to 5.0% w/w); or other compound substantially as described above or below. In pharmaceutical composition is surprisingly effective in instances where the subject is at risk of suffering from dry eye disease.

In still another related embodiment, a post-surgical treatment method is provided, which includes topically administering to an eye of a subject that has undergone ocular surgery, a therapeutically effective amount of a pharmaceutical composition, which includes mycophenolic acid at a concentration from 0.05% to 0.50% w/w and a pharmaceutically acceptable carrier for topical administration to the eye. In some embodiments, the pharmaceutical composition consists essentially of the mycophenolic acid, and the pharmaceutically acceptable carrier, preferably including a lubricant, for topical administration to the eye. In other embodiments, the pharmaceutical composition includes one or more of a glycosaminoglycan (e.g. chondroitin sulfate from 0.1% to 5.0% w/w); a deturgescent agent (e.g. dextran from 0.1% to 5.0% w/w); or other compound substantially as described above or below. In pharmaceutical composition is surprisingly effective in instances where the subject is at risk of suffering from dry eye disease

In addition, the pharmaceutical compositions described hereinabove may be useful for preventative and therapeutic treatment of other ophthalmic conditions and diseases as they are expected to provide numerous medical benefits such as for ocular surface (e.g., cornea and conjunctiva) lubrication, corneal deturgescence, cell membrane stabilization, etc. The ophthalmic compositions described hereinabove may be further useful for protecting the ocular surface, corneal epithelial cells, corneal endothelial cells, and/or other ocular tissues during an eye surgery. In addition, the ophthalmic compositions may be useful in wound healing after various injuries to the eye, for reducing corneal edema (e.g., during and after corneal transplantation surgery), for rehabilitating the ocular surface before and after contact lens wear, etc.

Pharmaceutical formulations described herein are preferably delivered topically, e.g., via eye drops. An ordinarily skilled physician may prescribe delivery by any other acceptable method if so desired and indicated, for example, by ophthalmic gel or ointment.

More specifically, the pharmaceutical compositions described hereinabove may be administered as a single dosage, in periodic applications, or may be maintained on the ophthalmic tissue continuously or substantially continuously as appropriate for the particular use. For example, they may be administered twice per day (BID), once per day, or once every minute for a period of 5 to 10 minutes, or more frequently, or less frequently. To illustrate, an effective amount of the pharmaceutical composition may be applied between 1 to 16 times a day (e.g., from 1 to 8 times per day, from 1 to 6 times per day, or from 1 to 4 times per day), or more frequently, or less frequently, as needed.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular disease or condition being treated.

In additional embodiments, pharmaceutical kits are provided. The kit includes a sealed container approved for the storage of pharmaceutical compositions, the container containing one of the above-described pharmaceutical compositions. An instruction for the use of the composition and the information about the composition are to be included in the kit. Exemplary sealed containers useful in the kits include, but are not limited to, reusable or disposable storage bottles, resealable or disposable foil pouches, etc.

The following examples are provided to further elucidate the advantages and features of the present invention, but are not intended to limit the scope of the invention. The example is for the illustrative purposes only. USP pharmaceutical grade products were used in preparing the formulations described below.

### C. Examples

### Example 1. Preparing a Pharmaceutical Composition No. 1

A pharmaceutical composition was prepared as described below. The following products were used in the amounts specified:
(a) about 0.10 g of betamethasone sodium phosphate;
(b) about 0.25 g of chondroitin sulfate (bovine);
(c) about 0.10 g of powdered edetate disodium dehydrate;
(d) about 0.2 g of Pluronic® F-127;
(e) about 1.0 mL of glycerol;
(f) about 0.125 g of METHOCEL® E4M; and
(g) about 100 mL of balanced salt solution.

Betamethasone sodium phosphate, chondroitin sulfate, edetate disodium dehydrate, and PLURONIC® F-127 were combined with about 90% of the balanced salt solution and stirred until completely dissolved. With continued stirring, METHOCEL® E4M was added followed by adding glycerol. The pH of the solution was then adjusted to about 6.8-7.2 using sodium hydroxide solution and the remainder of the balanced salt solution was added. The solution was then filtered through a 0.2 micron filter into a sterile droptainer.

### Example 2. Preparing a Pharmaceutical Composition No. 2

A pharmaceutical composition was prepared as described below. The following products were used in the amounts specified:
(a) about 0.268 g of mycophenolate sodium powder;
(b) about 0.25 g of chondroitin sulfate (bovine);
(c) about 0.10 g of powdered edetate disodium dehydrate;
(d) about 0.2 g of PLURONIC® F-127;
(e) about 1.0 mL of glycerol;
(f) about 0.125 g of METHOCEL® E4M; and
(g) about 100 mL of balanced salt solution.

Mycophenolate sodium, chondroitin sulfate, edetate disodium dehydrate, and PLURONIC® F-127 were combined with about 90% of the balanced salt solution and stirred until completely dissolved. With continued stirring, METHOCEL® E4M was added followed by adding glycerol. The pH of the solution was then adjusted to about 7.3-7.4 using sodium hydroxide solution and the remainder of the balanced salt solution was added. The solution was then filtered through a 0.2 micron filter into a sterile droptainer.

### Example 3. Preparing a Pharmaceutical Composition No. 3

A pharmaceutical composition was prepared as described below. The following products were used in the amounts specified:
(a) about 0.10 g of betamethasone sodium phosphate;
(b) about 0.25 g of chondroitin sulfate (bovine);
(c) about 0.25 of powdered dextran 40,000;
(d) about 0.10 g of powdered edetate disodium dehydrate;
(e) about 0.20 g of PLURONIC® F-127;
(f) about 1.0 mL of glycerol;
(g) about 0.10 g of METHOCEL® E4M; and
(h) about 100 mL of balanced salt solution.

Betamethasone sodium phosphate, chondroitin sulfate, dextran, edetate disodium dehydrate, and PLURONIC® F-127 were combined with about 90% of the balanced salt solution and stirred until completely dissolved. With continued stirring, METHOCEL® E4M was added followed by adding glycerol. The pH of the solution was then adjusted to about 6.8-7.2 using sodium hydroxide solution and the remainder of the balanced salt solution was added. The solution was then filtered through a 0.2 micron filter into a sterile droptainer.

### Example 4. Preparing a Pharmaceutical Composition No. 4

A pharmaceutical composition was prepared as described below. The following products were used in the amounts specified:
(a) about 0.535 g of mycophenolate sodium powder;
(b) about 0.25 g of chondroitin sulfate (bovine);
(c) about 0.25 of powdered dextran 40,000;
(d) about 0.30 g of powdered sodium thiosulfate pentahydrate;
(e) about 0.20 g of PLURONIC® F-127;
(f) about 1.0 mL of glycerol;
(g) about 0.10 g of METHOCEL® E4M;
(h) about 40 mL of balanced salt solution; and
(i) about 100 mL of sterile injectable water.

Chondroitin sulfate, dextran, sodium thiosulfate, and PLURONIC® F-127 were combined with of the balanced salt solution and with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly followed by adding METHOCEL® E4M and adjusting pH to about 7.3-7.4 and the remainder of water was added. The solution was then filtered through a 0.2 micron filter into a sterile droptainer.

### Example 5. Preparing a Pharmaceutical Composition No. 5

A pharmaceutical composition was prepared as described below. The following products were used in the amounts specified:
(a) about 0.535 g of mycophenolate sodium powder;
(b) about 0.1g of betamethasone sodium phosphate powder;
(c) about 0.25 g of chondroitin sulfate (bovine);
(d) about 0.25 of powdered dextran 70,000;
(e) about 0.30 g of powdered sodium thiosulfate pentahydrate;
(f) about 0.20 g of PLURONIC® F-127;
(g) about 1.0 mL of glycerol;
(h) about 1.17 g of sodium phosphate dibasic anhydrous;
(i) about 0.14 g of sodium phosphate monobasic anhydrous;
(j) about 0.10 g of METHOCEL® E4M;
(k) about 40 mL of balanced salt solution; and
(l) about 100 mL of sterile injectable water.

Chondroitin sulfate, dextran, sodium thiosulfate, and PLURONIC® F-127 were combined with of the balanced salt solution and with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly followed by adding METHOCEL® E4M and adjusting pH to about 7.3-7.4 and the remainder of water was added. The solution was then filtered through a 0.2 micron filter into a sterile droptainer.

### Example 6. Preparing a Pharmaceutical Composition No. 6

A pharmaceutical composition was prepared as described below. The following products were used in the amounts specified:
(a) about 0.535 g of mycophenolate sodium powder;
(b) about 0.031g of tacrolimus monohydrate powder;
(c) about 0.25 g of chondroitin sulfate (bovine);
(d) about 0.25 of powdered dextran 70,000;
(e) about 0.1 g of edetate disodium powder;
(f) about 0.30 g of powdered sodium thiosulfate pentahydrate;
(g) about 0.20 g of PLURONIC® F-127;
(h) about 1.0 mL Polysorbate 80;
(i) about 4.0 mL polyethylene glycol 400 MW;
(j) about 1.0 mL of glycerol;
(k) about 1.17 g of sodium phosphate dibasic anhydrous;
(l) about 0.14 g of sodium phosphate monobasic anhydrous;
(m) about 0.10 g of METHOCEL® E4M;
(n) about 40 mL of balanced salt solution; and
(o) about 100 mL of sterile injectable water.

Chondroitin sulfate, dextran, sodium thiosulfate, and PLURONIC® F-127 were combined with of the balanced salt solution and with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium and tacrolimus monohydrate were added slowly followed by adding METHOCEL® E4M and adjusting pH to about 7.3-7.4 and the remainder of water was added. The solution was then filtered through a 0.2 micron filter into a sterile droptainer.

### Example 7. Preparing a Pharmaceutical Composition No. 7

A pharmaceutical composition was prepared as described below. The following products were used in the amounts specified:
(a) about 0.321 g of mycophenolate sodium powder;
(b) about 0.01g of betamethasone sodium phosphate powder;
(c) about 0.25 g of chondroitin sulfate (bovine);
(d) about 0.25 of powdered dextran 70,000;
(e) about 0.30 g of powdered sodium thiosulfate pentahydrate;
(f) about 0.20 g of PLURONIC ® F-127;
(g) about 1.0 mL of glycerol;
(h) about 1.17 g of sodium phosphate dibasic anhydrous;
(i) about 0.4 g of sodium phosphate monobasic anhydrous;
(j) about 0.10 g of METHOCEL® E4M;
(k) about 40 mL of balanced salt solution; and
(l) about 100 mL of sterile injectable water.

Chondroitin sulfate, dextran, sodium thiosulfate, and PLURONIC® F-127 were combined with 90% of the balanced salt solution and about 90% of the water and stirred until completely dissolved followed by addition of glycerol with continued stirring. The pH of the solution was then adjusted to about 7.0 using sodium hydroxide solution before introducing mycophenolate sodium. A stock solution of 1% betamethasone sodium phosphate was prepared by dissolving 1 gm of betamethasone sodium phosphate powder in 100 mL water and confirming the stock solution concentration by HPLC. 1 ml of the 1% betamethasone sodium phosphate stock solution was then added to the mixture with continued stirring.

With continued stirring, mycophenolate sodium was added slowly followed by addition of METHOCEL® E4M, adjusting pH to about 7.3-7.4, and addition of the remainder of the water. The solution was then filtered through a 0.2 micron filter into sterile dropper bottles or unit dose vials.

### Example 8. Preparing a Pharmaceutical Composition Nos. 8 and 9

Pharmaceutical compositions were prepared as described below. The following products were used in the amounts specified:
(a) about 0.1 g (Composition 8) or 0.3 g (Composition 9) of mycophenolate sodium powder;
(b) about 0.25 g of chondroitin sulfate (bovine);
(c) about 0.25 of powdered dextran 70,000;
(d) about 0.1 g of edetate disodium powder;
(e) about 0.30 g of powdered sodium thiosulfate pentahydrate;
(f) about 0.03 g potassium chloride;
(g) about 0.20 g of PLURONIC® F-127;
(h) about 0.2 g of glycerol;
(i) about 0.98 g of sodium phosphate dibasic anhydrous;
(j) about 0.18 g of sodium phosphate monobasic anhydrous;
(k) about 100 mL of sterile injectable water.

Chondroitin sulfate, dextran, sodium thiosulfate, potassium phosphate, dibasic and monobasic sodium phosphate and PLURONIC® F-127 were combined with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly and adjusting pH to about 7.0-7.4 and the remainder of water was added. Additional, sodium hydroxide may be added after the mycophenolate sodium to obtain the final pH, if necessary. The solution was then filtered through a 0.2 micron filter into a sterile droptainer.

### Example 9. Preparing a Pharmaceutical Composition Nos. 10 and 11

A pharmaceutical composition was prepared as described below. The following products were used in the amounts specified:
(a) about 0.02 g (Composition 10) or 0.04 g (Composition 11) of betamethasone sodium phosphate;
(b) about 0.25 g of chondroitin sulfate (bovine);
(c) about 0.25 of powdered dextran 70,000;
(d) about 0.1 g of edetate disodium powder;
(e) about 0.30 g of powdered sodium thiosulfate pentahydrate;
(f) about 0.03 g potassium chloride;
(g) about 0.20 g of PLURONIC® F-127;
(h) about 0.1 g of glycerol;
(i) about 0.98 g of sodium phosphate dibasic anhydrous;
(j) about 0.18 g of sodium phosphate monobasic anhydrous;
(k) about 100 mL of sterile injectable water.

Betamethasone sodium phosphate, dextran, sodium thiosulfate, potassium chloride, dibasic and monobasic sodium phosphate and PLURONIC® F-127 were combined with about 90% of water and stirred until completely dissolved followed by adding glycerol with continued stirring. The pH of the solution was then adjusted to about 7.0-7.4 using sodium hydroxide solution. The solution was then filtered through a 0.2 micron filter into a sterile droptainer.

### Example 10. Preparing a Pharmaceutical Composition No. 12

A pharmaceutical composition was prepared as described below. The following products were used in the amounts specified:
(a) about 0.02 g to 0.10 g of betamethasone sodium phosphate;
(b) about 0.1% mycophenolate sodium;
(c) about 0.25 g of chondroitin sulfate (bovine);
(d) about 0.25 of powdered dextran 70,000;
(e) about 0.1 g of edetate disodium powder;
(f) about 0.30 g of powdered sodium thiosulfate pentahydrate;
(g) about 0.03 g potassium chloride;
(h) about 0.20 g of PLURONIC® F-127;
(i) about 0.1 g of glycerol;
(j) about 0.98 g of sodium phosphate dibasic anhydrous;
(k) about 0.18 g of sodium phosphate monobasic anhydrous;
(l) about 100 mL of sterile injectable water.

Betamethasone sodium phosphate, sodium mycophenolate, dextran, sodium thiosulfate, potassium chloride, dibasic and monobasic sodium phosphate and PLURONIC® F-127 were combined with about 90% of water and stirred until completely dissolved followed by adding glycerol with continued stirring. The pH of the solution was then adjusted to about 7.0-7.4 using sodium hydroxide solution. The solution was then filtered through a 0.2 micron filter into a sterile droptainer.

Although the invention has been described with the reference to the above examples, it will be understood that modifications and variations are encompassed within the spirit and scope of the invention. Accordingly, the invention is limited only by the following claims.

What is claimed is:

1. A pharmaceutical composition for topical administration to an eye, the composition comprising betamethasone sodium phosphate at a concentration of 0.01% w/w to 0.05% w/w; mycophenolic acid at a concentration of 0.05% w/w to 0.50% w/w; and a pharmaceutically acceptable carrier for topical administration to the eye.

2. The pharmaceutical composition of claim 1, further comprising a glycosaminoglycan.

3. The pharmaceutical composition of claim 2, wherein the glycosaminoglycan comprises chondroitin sulfate.

4. The pharmaceutical composition of claim 3, wherein the chondroitin sulfate is at a concentration of 0.1% w/w to 5.0% w/w.

5. The pharmaceutical composition of claim 3, further comprising a deturgescent agent.

6. The pharmaceutical composition of claim 5, wherein the deturgescent agent comprises dextran.

7. The pharmaceutical composition of claim 6, wherein the dextran is at a concentration of 0.1% w/w to 5.0% w/w.

8. The pharmaceutical composition of claim 1, wherein the composition consists essentially of betamethasone sodium phosphate at a concentration of 0.01% w/w to 0.05% w/w; mycophenolic acid at a concentration of 0.05% w/w to 0.50% w/w; and a pharmaceutically acceptable lubricating carrier for topical administration to the eye.

9. A post-surgical ocular treatment method comprising: topically administering to an eye of a subject that has undergone ocular surgery, a therapeutically effective amount of the pharmaceutical composition of claim 1.

10. The method of claim 9, wherein the pharmaceutical composition further comprises a chondroitin sulfate.

11. The method of claim 10, wherein the pharmaceutical composition further comprises dextran.

12. The method of claim 9, wherein the subject suffers from or is at risk of suffering from dry eye disease.

13. A post-surgical ocular treatment method comprising: topically administering to an eye of a subject that has undergone ocular surgery, a therapeutically effective amount of the pharmaceutical composition of claim 8.

14. The method of claim 13, wherein the subject suffers from or is at risk of suffering from dry eye disease.

15. A post-surgical ocular treatment method comprising: topically administering to an eye of a subject that has undergone ocular surgery, a therapeutically effective amount of a pharmaceutical composition comprising betamethasone sodium phosphate at a concentration of 0.01% w/w to 0.05% w/w.

16. The method of claim 15, wherein the pharmaceutical composition consists essentially of the betamethasone sodium phosphate and a pharmaceutically acceptable lubricating carrier for topical administration to the eye.

17. The method of claim 15, wherein the subject suffers from or is at risk of suffering from dry eye disease.

18. A method of treating dry eye disease, the method comprising: administering to a subject suffering from dry eye disease, a therapeutically effective amount of the pharmaceutical composition of claim 1.

19. The method of claim 18, wherein the pharmaceutical composition further comprises a chondroitin sulfate.

20. The method of claim 19, wherein the pharmaceutical composition further comprises dextran.

21. A method of treating dry eye disease, the method comprising: administering to a subject suffering from dry eye disease, a therapeutically effective amount of the pharmaceutical composition of claim 8.

22. A method of treating dry eye disease, the method comprising: administering to a subject suffering from dry eye disease, a therapeutically effective amount of a pharmaceutical composition comprising betamethasone sodium phosphate at a concentration of 0.01% w/w to 0.05% w/w.

23. The method of claim 22, wherein the pharmaceutical composition consists essentially of the betamethasone sodium phosphate and a pharmaceutically acceptable lubricating carrier for topical administration to the eye.

24. A method of treating dry eye disease, the method comprising: administering to a subject suffering from dry eye disease, a therapeutically effective amount of a pharmaceutical composition comprising mycophenolic acid at a concentration of 0.05% w/w to 0.50 w/w.

25. The method of claim 24, wherein the pharmaceutical composition consists essentially of the mycophenolic acid and a pharmaceutically acceptable lubricating carrier for topical administration to the eye.
